(19) United States
(12) Patent Application Publication
(10) Pub. No.: US 2024/0108658 A1
(43) Pub. Date: Apr. 4, 2024

(54) SYNERGISTIC STEM CELL-BASED MEDICATION FOR TREATING MYOCARDIAL INFARCTION AND RELATED APPLICATION

(71) Applicant: SOOCHOW UNIVERSITY, Suzhou (CN)

(72) Inventors: Zhenya SHEN, Suzhou (CN); Weiqian CHEN, Suzhou (CN); Weizhang XIAO, Suzhou (CN); Ming CHEN, Suzhou (CN)

(21) Appl. No.: 18/035,685

(22) PCT Filed: Jun. 23, 2021
(86) PCT No.: PCT/CN2021/101922

§ 371 Date: May 5, 2023

(51) Int. Cl. A61K 35/28; A61K 31/7004; A61P 9/10

(52) U.S. Cl. CPC A61K 35/28; A61K 31/7004; A61P 9/10

(57) ABSTRACT

The present invention discloses a drug for the augmentation treatment of myocardial infarction with stem cells and its application, specifically the application of 2-deoxy-D-glucose in the preparation of drugs for the augmentation treatment of myocardial infarction with stem cells. Animal experiments have confirmed that the injection of 2-deoxy-D-glucose increased the survival rate of injected mesenchymal stem cells and enhanced the therapeutic effect of mesenchymal stem cells.

### TECHNICAL FIELD

The present invention belongs to pharmaceutical technology and relates to a drug for the augmentation treatment of myocardial infarction with stem cells and its application, specifically the application of 2-deoxy-D-glucose in the preparation of drugs for the augmentation treatment of myocardial infarction with stem cells.

### BACKGROUND OF INVENTION

Myocardial infarction (MI) is a cardiovascular disease that seriously endangers human health. With the continuous improvement of our people's living standards, the incidence rate of ischemic myocardial infarction has also been rising. Ischemic myocardial infarction can lead to myocardial cell necrosis and scarring formation, thereby affecting cardiac function. Currently, the medication or device therapy can only alleviate symptoms, but it cannot reverse cardiac tissue damage. Although heart transplantation can completely improve heart condition, it is difficult to be widely applied clinically due to factors of scarce donor sources, immune rejection, and expensive treatment costs.

Stem cells are a series of undifferentiated cells with the ability to be differentiated into specific cell types. Stem cells can be divided into embryonic stem cells (ESC) and adult stem cells according to the source. The latter includes hematopoietic stem cells (HSCs) and mesenchymal stem cells (MSCs). Mesenchymal stem cells exist in almost all tissues of the human body with specific stem cell niches. And the bone marrow-derived mesenchymal stem cells (BM-MSCs) are the first discovered mesenchymal stem cells and the most commonly used stem cells for the treatment of myocardial infarction. However, existing technology suggests that the efficacy of treating myocardial infarction only by stem cells still requires improvement.

### Technical Problems

2-Deoxy-D-glucose (2-DG) is a glucalogue that has the ability to interfere with the synthesis of specific viral glycoproteins and inhibit the proliferation of herpes simplex virus, RAN and DNA enveloped virus, and cancer cells; but so far, there have been no research reports on the role of 2-deoxy-D-glucose in treating myocardial infarction, especially the relationship between 2-DG and stem cells.

The present invention discloses a new use of 2-deoxy-D-glucose to improve the therapeutic effect of stem cells after myocardial infarction. The biggest problem with MSCs in treating myocardial infarction is the low retention rate and poor survival of transplanted cells at the site of the myocardial infarction. The present invention discloses a significant improvement in the therapeutic effect of 2-deoxy-D-glucose on mesenchymal stem cells after myocardial infarction.

### Technical Solution

The present invention adopts the following technical scheme: the application of 2-deoxy-D-glucose in the preparation of drugs for the augmentation treatment of myocardial infarction with stem cells.

The application of 2-deoxy-D-glucose in the preparation of medicine for treating myocardial infarction.

The application of 2-deoxy-D-glucose and stem cells in the preparation of drugs for the augmentation treatment of myocardial infarction.

A drug for the augmentation treatment of myocardial infarction has the active ingredients of the drug is 2-deoxy-D-glucose and stem cells. It also discloses the application of the drugs for the augmentation treatment of myocardial infarction in the preparation of drugs for the treatment of myocardial infarction.

The present invention relates to the application of 2-DG in the preparation of drugs or health care products for preventing or treating myocardial infarction, specifically, 2-DG can be used to prepare myocardial infarction protective drugs, health care products or foods, achieving the technical effect of 2-deoxy-D-glucose, especially augmentation stem cells, particularly mesenchymal stem cells to improve cardiac function, slow down ventricular dilation, and reduce myocardial infarction area after myocardial infarction.

The prepared drug disclosed in the present invention can be administered by intraperitoneal injection, etc. And the dosage of 2-deoxy-D-glucose is 300-800 mg/kg/day, such as 500 mg/kg/day.

### Beneficial Effects

Considering the success rate of MSCs transplantation, the MI local myocardial transplantation may be superior to intravenous injection or coronary artery transplantation, but regardless of the transplantation way used, the ultimate goal is still to optimize the strategy to enhance the survival rate of injected MSCs. Under the prior art, due to the complexity of the heart environment caused by MI, the effectiveness of the injection treatment of mesenchymal stem cells for myocardial infarction is not satisfactory. Researchers have adopted various methods to improve its therapeutic effect on myocardial infarction, such as in vitro culture of mesenchymal stem cells with cytokines and reinfusion, which is a complex operation. Myocardial infarction (MI) is a cardiovascular disease that seriously endangers human health. With the continuous improvement of our people's living standards, the incidence rate of ischemic myocardial infarction has also been rising. The present invention uses 2-deoxy-D-glucose for the first time to improve cardiac function after myocardial infarction, especially by combining with mesenchymal stem cells to enhance the therapeutic effect of stem cells for myocardial infarction and reduce the myocardial infarction area, thereby preventing or treating myocardial infarction.

### BRIEF DESCRIPTION OF THE DRAWINGS

FIG. 1 is the improvement of cardiac function of mice treated with MSCs after myocardial infarction using 2-DG.

FIG. 2 is the improvement of ventricular remodeling of mice treated with MSCs after myocardial infarction using 2-DG.

FIG. 3 shows that 2-DG significantly increases the survival of transplanted MSCs.

### EXAMPLES OF THE PRESENT INVENTION

The examples listed below are only intended to help those skilled in the art have a more comprehensive understanding of the present invention, but do not limit the present invention in any way. The raw materials, reagents, modeling, and testing involved in the present invention are conventional techniques in this field.

The main materials and sources used in the present invention are as follows: C57BL/6J mice (provided by Joinn Laboratories (Suzhou) Co., Ltd., and this experiment was approved by the Ethics Committee of Soochow University); small animal ventilator (Shanghai Alcott Biotech Co., Ltd., Shanghai); surgical instruments (66 Vision-Tech, Suzhou); suture needle and thread (Shanghai Pudong Jinhuan Medical Products Co., Ltd., Shanghai); small animal ultrasonic imaging system (Visual Sonics Vevo 2100); mesenchymal stem cells (MSCs) were extracted from the femoral bone marrow of 8-week-old male C57BL/6J mice using existing techniques, and were routinely cultured for use in experiments after the sixth generation; 2-deoxy-D-glucose (2-DG, Suzhou Tianke Trading Co., Ltd., Suzhou), dissolve in physiological saline (concentration 31.25 mg/mL) for backup.

Establishment of a mouse myocardial infarction modelling: C57BL/6J male mice with a weight of about 25 g were selected as the experimental subjects, and the myocardial infarction modelling was established by using the ligation of left anterior descending coronary artery. After intraperitoneal injection anesthesia, an oral endotracheal intubation was performed and an air ventilator was connected. The respiratory rate was 110 times/min, the tidal volume was 2.5 ml, and the inspiration and expiration ratio was 1:1.3. The mice lied on the right side, the outer skin of the left chest was incised with a longitudinal incision, the pectoralis major was peeled off, the chest was opened through a transverse incision between the third and fourth ribs to expose the heart and pericardium was torn with forceps. The rough distribution of left coronary artery can be seen with the help of a surgical microscope. The left anterior descending (LAD) and a small amount of myocardial tissue were ligated about 1.5 mm of the lower edge of the left atrial appendage with needle depth of about 1 mm and width of within 3 mm; and the chest was closed layer by layer.

The sham-operated group was only passed through the lower part of the LAD without ligation, and the rest were the same as the model group.

After ligation, it turned white from the ligation site to the cardiac apex when observed with the naked eyes. The left ventricular tissue was taken for cardiac tissue staining after 1 week and obvious fibrosis was observed, indicating the successful establishment of the myocardial infarction modelling.

The MSCs group was injected with extracted MSCs from three points at the edge of the myocardial infarction immediately after LAD ligation, with a total number of injected cells of 5×105/20 μl PBS; the 2-DG group only received intraperitoneal injection of 2-DG 6 hours before the establishment of myocardial infarction, 1 day after myocardial infarction surgery, and 2 days after surgery (500 mg/kg/day); the MSCs+2-DG group received intraperitoneal injection of 2-DG 6 hours before the establishment of myocardial infarction, 1 day after myocardial infarction surgery, and 2 days after surgery (500 mg/kg/day), while the rest were the same as the MSCs group; the control group (saline group) mice were injected with an equal amount of sterile saline after LAD ligation as a control; the feeding methods of the three groups were the same, and then cardiac function and myocardial infarction area were tested after myocardial infarction.

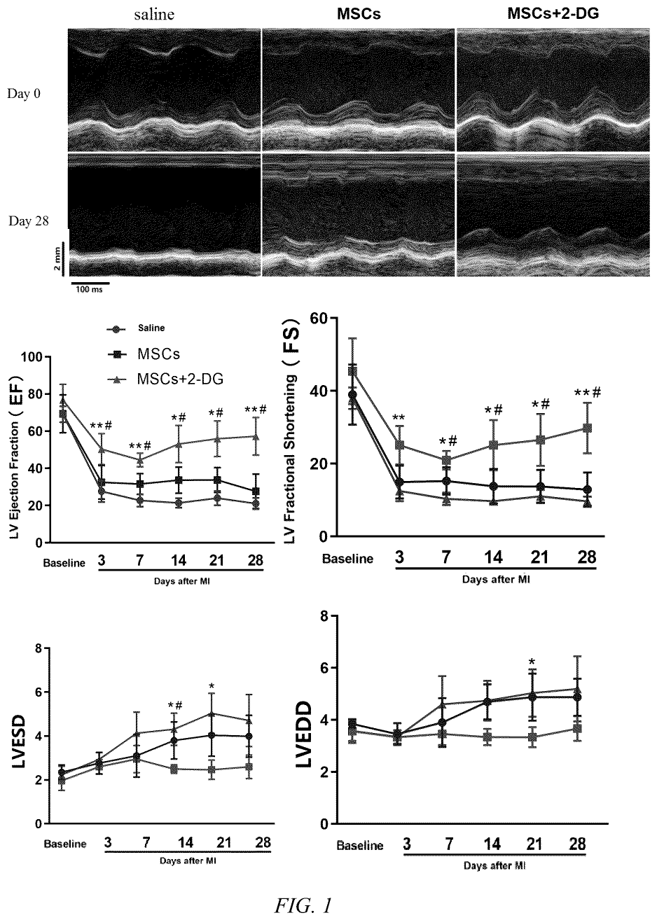

Example 1: Effective improvement of cardiac function after myocardial infarction: The cardiac ultrasound testing of cardiac function after myocardial infarction. After anesthesia of mice (same method as before) and depilation, the mice lied on the left side, and the probe of the cardiac ultrasound diagnostic instrument was placed on the anterior wall of the heart, a two-dimensional short-axis view of the left ventricle at the papillary muscle level was taken and the M-mode ultrasonic scanning was recorded. And left ventricular ejection fraction (EF), shortening fraction (FS), left ventricular end diastolic diameter (LVEDD), and left ventricular end systolic diameter (LVESD) were measured for three consecutive cardiac cycles. The experimental results were shown in FIG. 1, *P<0.05; #P<0.05.

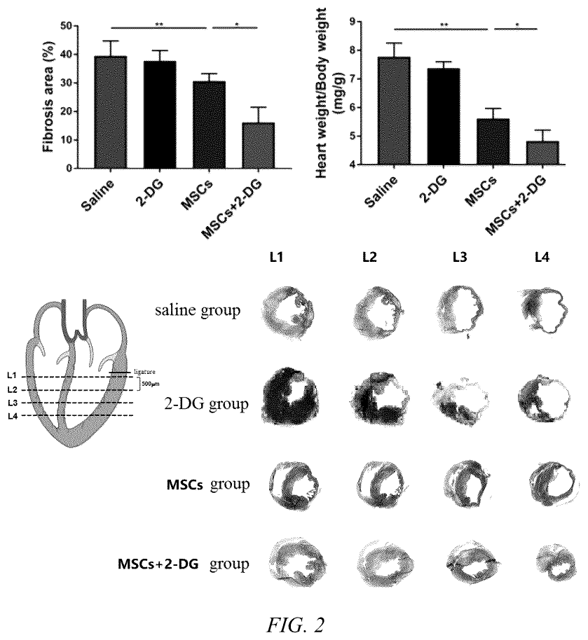

Example 2: Effective improvement of ventricular remodeling: Masson staining for heart weight: The mice were euthanized 28 days after surgery, and their body weight (BW) and heart weight (HW) were measured; HW/BW was calculated. Masson staining: The mice were euthanized 28 days after surgery, and left ventricular tissue was taken for cardiac tissue staining to observe the therapeutic effect. It was carried out according to the routine Masson staining steps, and the observation and photo-taking were carried out under a regular optical microscope. The image analysis software Image J was used to analyze the area of each part and calculate the myocardial infarction area/heart area. The experimental results were shown in FIG. 2, *P<0.05; **P<0.01.

Example 3: Increase of survival of mesenchymal stem cells: The mesenchymal stem cells stained with CM-Dil were prepared and the survival rate of transplanted mesenchymal stem cells was observed with the red fluorescence characteristics of CM-Dil. Specifically, the CM-Dil staining solution was prepared: The concentration of the CM-Dil dye storage solution (Thermo Fisher) was 1 mg/mL, and 4 uL of the storage solution was added to 2 mL of PBS to achieve a final concentration of 2 ug/mL. The MSCs (500,000) to be injected were taken for conventional centrifugation, and then they were resuspended with CM-Dil staining solution. Then they were incubated at 37° C. for 5 min, and placed at 4° C. for 15 min, and then thoroughly washed with PBS 3 times and added with 20 uL PBS to be resuspended for intramyocardial injection after myocardial infarction.

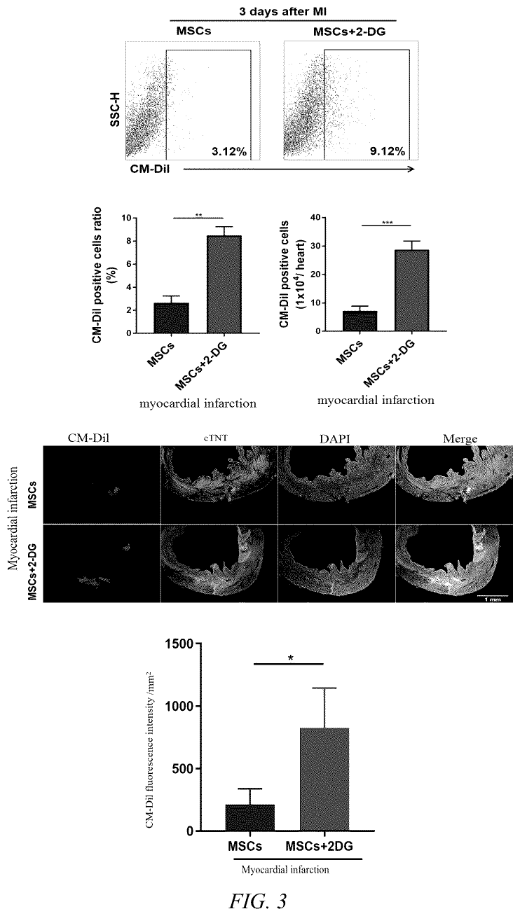

On the 3rd day after surgery, frozen sections of the heart were taken and tissue sections of troponin (cTNT) were prepared for immunofluorescence. Troponin (cTNT) was labeled in green, CM-Dil was labeled in red, and DAPI was labeled in blue; On the 3rd day after surgery, the heart was digested to prepare a single cell suspension, and the proportion and quantity of CM-Dil positive cells were detected by flow cytometry. The experimental results were shown in FIG. 3, *P<0.05.

### CONCLUSION

The above experimental results comprehensively prove that 2-DG can effectively improve the function of MSCs in improving myocardial infarction.

The above are only the preferred Examples of the present invention. It should be pointed out that for ordinary technical personnel in this field, several improvements and embellishments can be made without departing from the principles of the present invention. These improvements and embellishments should also be considered as the scope of protection of the present invention.

1. A drug for the augmentation treatment of myocardial infarction, characterized in that the active ingredients of the drug are 2-deoxy-D-glucose and stem cells.

2. The drug for the augmentation treatment of myocardial infarction according to the claim 1, characterized in that the drug is an injection preparation.

3. An application of 2-deoxy-D-glucose in the preparation of drugs for treating myocardial infarction with synergistic stem cells.

4. The application according to the claim 3, characterized in that the drug is an injectable preparation.

5. The application according to the claim 3, characterized in that 2-deoxy-D-glucose improves cardiac function and ventricular remodeling in stem cell treatment after myocardial infarction.

6. An application of 2-deoxy-D-glucose in the preparation of drugs for the treatment of myocardial infarction; of 2-deoxy-D-glucose and stem cells in the preparation of drugs for the augmentation treatment of myocardial infarction; of 2-deoxy-D-glucose in the preparation of health care products for the prevention of myocardial infarction; or of 2-deoxy-D-glucose in the preparation of drugs that increase the survival rate of stem cells in myocardial infarction sites.

7. (canceled)

8. (canceled)

9. (canceled)

10. The application of the drugs for the augmentation treatment of myocardial infarction according to the claim 1 in the preparation of drugs for the treatment of myocardial infarction.
